(19) United States
(12) Patent Application Publication
(10) Pub. No.: US 2017/0248572 A1
(43) Pub. Date: Aug. 31, 2017

(54) LUBRICANT CONDITION ASSESSMENT SYSTEM

(71) Applicant: Sikorsky Aircraft Corporation, Stratford, CT (US)

(72) Inventors: Carl S. Byington, Pittsford, NY (US); Carl Palmer, Pittsford, NY (US); Cody Michael Ture, Fairport, NY (US); David Ortiz, Rochester, NY (US); Ryan Brewer, Rochester, NY (US); John R. Farnach, Henrietta, NY (US); Christopher M. Minnella, Pittsford, NY (US)

(21) Appl. No.: 15/514,581

(22) PCT Filed: Jul. 24, 2015
(86) PCT No.: PCT/US15/41933

§ 371 Date: Mar. 27, 2017

Related U.S. Application Data
(60) Provisional application No. 62/055,899, filed on Sep. 26, 2014.

(51) Int. Cl. G01N 33/28; F16H 57/04; G01N 33/30; G01N 27/07; G01N 11/00

(52) U.S. Cl. CPC G01N 33/2888; G01N 27/07; G01N 11/00; G01N 33/30; F16H 57/0405

(57) ABSTRACT

An apparatus for assessment of a fluid system includes a debris monitor to receive a first flow of a fluid, the debris monitor to determine wear debris information in the first flow of the fluid; and a fluid condition monitor to receive a second flow of the fluid, the fluid condition monitor being configured to determine fluid condition information in the second flow of the fluid.

### STATEMENT REGARDING FEDERALLY SPONSORED RESEARCH OR DEVELOPMENT

This invention was made with Government support with the United States Army under Contract No. W911W6-09-C-0049. The Government therefore has certain rights in this invention.

### BACKGROUND

The subject matter disclosed herein relates generally to the field of fluid analysis and, more particularly, to a lubricant condition assessment system that integrates lubricant quality assessment and debris monitoring into an integrated package.

### DESCRIPTION OF RELATED ART

Aircraft mechanical components require wear protection fluids such as drive train lubricants and engine oils to keep the aircraft components operating in the most efficient and safe manner possible. Lubricating fluids can become degraded or contaminated by internal or external sources or accumulate component wear debris due to pitting, spalling, corrosion-induced fatigue, or other mechanisms. Further, water infiltration or chemical changes can degrade the lubricant and can affect oil-wetted component lifetimes and maintenance requirements.

Lubricant monitoring of oil-wetted mechanical components is being widely used for diagnostic and prognostic assessment of the health of these mechanical components. Two typical lubricant monitoring techniques include lubricant analysis and detection of metallic debris suspended in lubricant flow. Lubricant analysis is typically performed off-line and may include lab analysis and optical inspection with a sample of lubricant from the system whose condition is to be assessed. The off-line lubricant analysis can be slow, labor intensive, expensive and error prone. On the other hand, metallic debris monitoring is found as an online capability and can include a chip detector (magnetic plug) to collect ferrous materials for analysis and inspection. However, this metallic debris monitoring is not sensitive to detecting non-ferrous debris such as magnesium alloys or aluminum alloys. Typically, these two monitoring techniques are most commonly performed separately as they involve different technologies and processes. A sensing system that integrates a lubrication condition monitor with wear debris detection for lubricant-wetted mechanical systems would be highly beneficial in the art.

### BRIEF SUMMARY

According to an aspect of the invention, an apparatus for assessment of a fluid system includes a debris monitor to receive a first flow of a fluid, the debris monitor being configured to determine wear debris information in the first flow of the fluid; and a fluid condition monitor to receive a second flow of the fluid, the fluid condition monitor being configured to determine fluid condition information in the second flow of the fluid.

In the above embodiment, or as an alternative, the debris monitor comprises a sensing element, the sensing element comprising one or more of an inductive coil, an optical sensing element, a magnetic sensing element, and an acoustical sensing element that obtains the wear debris information.

In the above embodiment, or as an alternative, the sensing element includes the inductive coil, the debris monitor to identify wear debris particles in the fluid by analyzing real and imaginary impedance shifts in magnetic and electric field lines.

In the above embodiment, or as an alternative, a communication controller is provided to provide communication of at least one of the wear debris information and the fluid condition information to an external interface.

In the above embodiment, or as an alternative, the first flow and the second flow are a same flow.

In the above embodiment, or as an alternative, the debris monitor and the fluid condition monitor are positioned in at least one of an in-line flow path, an on-line flow path and an off-line flow path.

In the above embodiment, or as an alternative, a housing is provided, the housing comprising a first flange at a first end and a second flange at a second end, the first flange to couple the housing to the fluid system, the second flange to couple a filter to the housing.

In the above embodiment, or as an alternative, a pathway to receive a third flow of the fluid is provided, the pathway including a particle capture element in fluidic communication with the third flow of the fluid, the particle capture element to provide a sample of wear debris particles in the third flow of the fluid.

In the above embodiment, or as an alternative, the third flow of the fluid is parallel to at least one of the first flow of the fluid and the second flow of the fluid.

In the above embodiment, or as an alternative, the fluid condition monitor is configured to determine at least one of water content, incorrect lubricant addition, lubricant oxidation degradation, additive depletion, or viscosity.

In the above embodiment, or as an alternative, the fluid is a lubricant from a gearbox of a vehicle.

In the above embodiment, or as an alternative, the fluid condition information comprises at least one of dielectric properties, conductivity, and fluid impedance.

In the above embodiment, or as an alternative, the debris monitor includes at least one of analog circuitry, an analog-to-digital converter, and digital processing circuitry.

In the above embodiment, or as an alternative, the fluid condition monitor includes at least one of analog circuitry, an analog-to-digital converter, and digital processing circuitry.

Other aspects, features and techniques of the invention will become more apparent from the following description taken in conjunction with the drawings.

### BRIEF DESCRIPTION OF THE SEVERAL VIEWS OF THE DRAWINGS

The subject matter, which is regarded as the invention, is particularly pointed out and distinctly claimed in the claims at the conclusion of the specification. The foregoing and other features, and advantages of the invention are apparent from the following detailed description taken in conjunction with the accompanying drawings in which like elements are numbered alike in the several FIGURES:

FIG. 1 is a view of an exemplary system in accordance with an embodiment of the invention;

FIG. 2A illustrates a cross-sectional view of a lubricant condition assessment apparatus in accordance with an embodiment of the invention;

FIG. 2B illustrates a front view of a lubricant condition assessment apparatus as shown in phantom in accordance with an embodiment of the invention;

FIG. 2C illustrates a rear cross-sectional view of a lubricant condition assessment apparatus in accordance with an embodiment of the invention;

FIG. 2D illustrates a rear view of a lubricant condition assessment apparatus as shown in phantom in accordance with an embodiment of the invention;

FIG. 3 depicts an exemplary plot of wear debris detection in accordance with an embodiment of the invention;

FIG. 4 depicts an exemplary plot of lubricant condition assessment in accordance with an embodiment of the invention; and

FIG. 5 depicts exemplary lubricant condition assessment topologies for use with the lubricant condition assessment apparatus.

### DETAILED DESCRIPTION

Exemplary embodiments are described with reference to a lubricant condition assessment system for use with a gearbox of a vehicle. It is understood that embodiments may more generally apply to a fluid condition assessment system for use with a variety of systems, such as hydraulic systems, coolant systems, etc. Therefore, although embodiments are described with reference to a lubricant condition assessment system, it is understood that embodiments of the invention are not intended to be limited to the analysis of lubricants, but may apply to a variety of fluids.

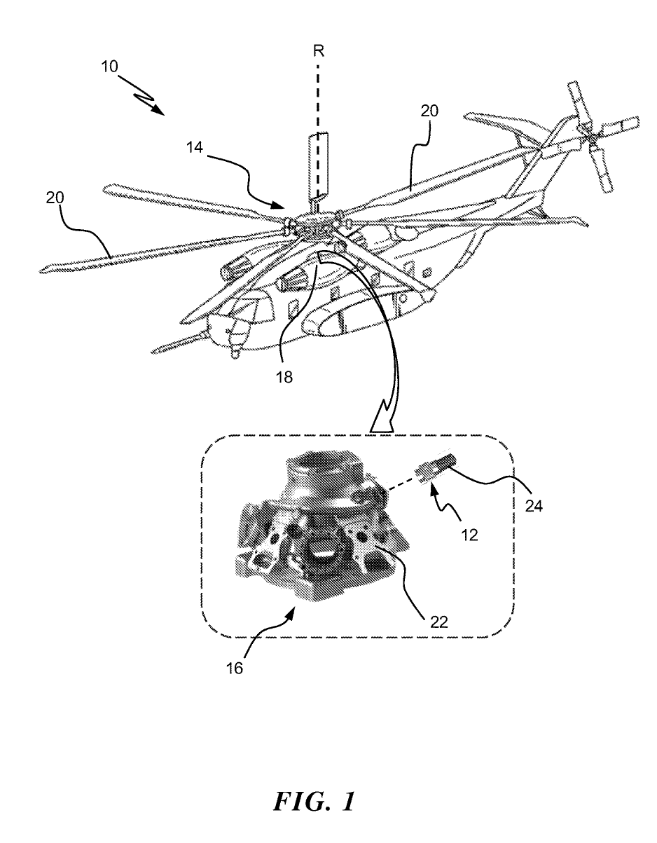

Referring to the drawings, FIG. 1 illustrates an exemplary vehicle with a gearbox, e.g., a helicopter or aircraft 10 having a gearbox 16 with a lubricant condition assessment apparatus 12 (hereinafter “LUCAS apparatus 12”) that provides lubrication condition assessment and wear debris detection of a lubricant in accordance with an embodiment of the invention. For clarity, lubricant can include oil, or other lubricating fluids. As shown, exemplary aircraft 10 includes a main rotor assembly 14 that is driven about an axis of rotation R by one or more engines 18. The main rotor assembly includes a multiple of rotor blades 20 mounted to rotor assembly 14 and are driven for rotation about axis R through a main gearbox 16. As illustrated, lubricant condition assessment system includes a LUCAS apparatus 12 that can be an embedded in-line, on-line or off-line, that integrates both lubricant condition monitoring and wear debris detection and captures information into a single, smart sensor device. LUCAS apparatus 12 can be positioned in-line with lubricant flow through main gearbox 16 and can be selectively coupled to housing 22 of main gearbox 16 and gearbox filter 24. As a result, LUCAS apparatus 12 provides in-line, real-time monitoring of lubricant as it travels from main gearbox 16, through LUCAS apparatus 12 and to filter 24. While LUCAS apparatus 12 is shown and described with an aircraft 10, LUCAS apparatus 12 may also be used to provide other vehicle gearboxes and engines with an in-line sensing solution for both characterization of lubricant condition (i.e., degradation and contamination) as well as the detection of wear debris including ferrous and non-ferrous materials generated by these gearboxes and other mechanical systems as they experience damage due to use, contaminate, or other root causes.

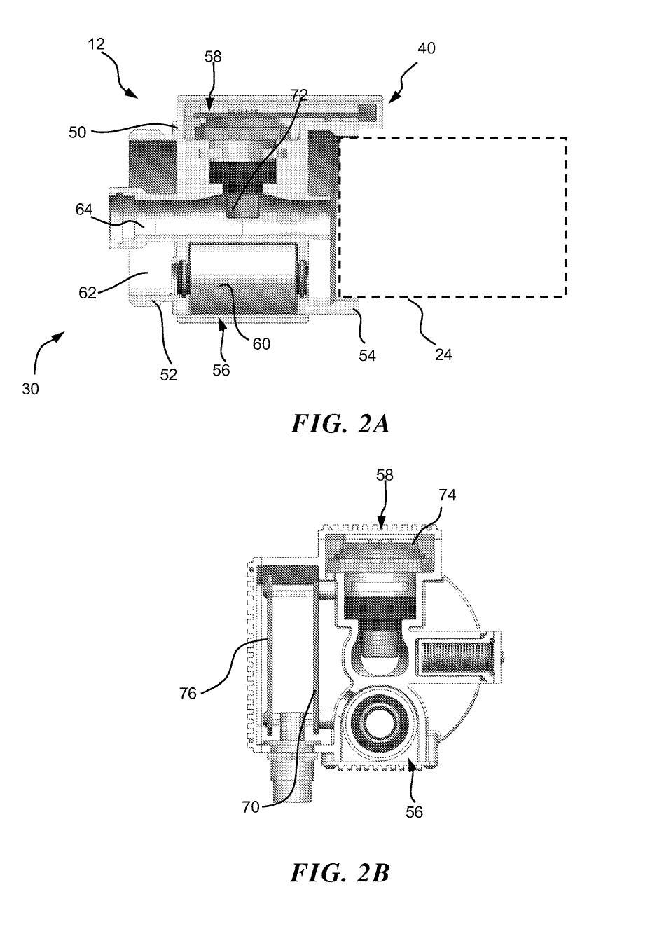

FIGS. 2A-2D depict an exemplary embodiment of LUCAS apparatus 12 as used on a gearbox of a vehicle, e.g., on main gearbox 16 of aircraft 10 in accordance with an embodiment of the invention. As illustrated, LUCAS apparatus 12 is configured to be positioned in-line with the flow of lubricant through a gearbox in order to provide a real time sensing solution for lubrication condition assessment and wear debris detection of ferrous and non-ferrous materials in the lubricant. LUCAS apparatus 12 includes housing 50 with an internal cavity that is configured to receive a debris monitor 56 and its associated debris controller 70, a fluid (e.g., lubricant) condition monitor 58 and its associated condition controller 74, and a communication controller 76.

In an embodiment, LUCAS apparatus 12 has a generally cylindrical housing 50 of unitary construction that can be cast from a metal or a metal alloy. Housing 50 includes a first flange 52 at a proximal end 30 and a second directionally opposite flange 54 at a distal end 40. Housing 50 is shown with a first flange 52 that includes external circumferential threads that are configured to be threadably coupled to complementary threads of a filter port of a gearbox, e.g., main gearbox 16 (FIG. 1) while second flange 54 can include internal circumferential threads that are configured to be threadably coupled to complementary threads of an external gearbox filter 24 (shown in phantom). In other embodiments, first flange 52 and second flange 54 can be coupled to their respective and complementary interfaces through other methods such as, for example, studs, pins, bolts, or the like. Housing 50 includes a bore 60 that channels contaminated or “dirty” lubricant from input chamber 62 of a gearbox 16 (FIG. 1) to gearbox filter 24 and a through-bore 64 that returns “filtered” lubricant from gearbox filter 24 to gearbox via housing 50 for oil condition assessment. For clarity purposes, contaminated or “dirty” lubricant includes lubricant that is received from gearbox 16 (FIG. 1) such as, for example, oil that is used for lubricating internal moving parts and gears in gearbox 16. An optional debris sample capture port 66 (shown in FIG. 2C-2D) can be provided in housing 50 to selectively provide a user with a coarse sample of wear debris particles that may be present in the flow of “dirty” lubricant as it traverses a bore 68. Bore 68 provides an alternate and parallel pathway for “dirty” lubricant to travel from gearbox to gearbox filter 24.

As shown in FIG. 2A, debris monitor 56 is generally tubular and surrounds bore 60. Debris monitor 56 may include one or more of a sensing element, analog circuitry, analog-to-digital converter(s), and/or digital processing circuitry. An exemplary sensing element is an inductive coil that surrounds bore 60 to create a magnetic field within the bore when excited by a high frequency alternating current. Sensing elements may include one or more of an inductive coil, an optical sensing element, magnetic sensing element, acoustical sensing element, etc.

The inductive coil detects wear debris particles in the lubricant by detecting the interaction between particles and the inductive coil. Debris controller 70 generates electric and magnetic fields in the inductive coil and includes a phase-sensitive demodulator for detecting real and imaginary impedance shifts in the bridge circuit caused by ferrous or non-ferrous wear debris particles. The electromagnetic inductance is represented in a real component of the sensed impedance signal and the magnetic flux reluctance is represented in the imaginary component of the sensed impedance signal. Ferrous and non-ferrous wear debris particles have different effects on the electric and magnetic fields of the inductive coil. As wear debris particles comprising ferrous and non-ferrous particles pass through bore 60, they modify the fields generated by the inductive coil, and produce unique signatures through coil imbalance that can be categorized based on the properties of the signal that is sensed. Also, a magnitude of the disruptive signal provides an approximate size of ferrous or non-ferrous particles in the lubricant flow. To detect a size and type of wear debris particles, debris monitor 56 includes a debris controller 70 housed within housing 50. Debris controller 70 may be implemented as a microcontroller, DSP, microprocessor or similar device and includes a memory. The memory may store a debris detection algorithm as executable instructions for identifying ferrous and non-ferrous wear debris particles and count of wear debris particles in the lubricant. Also, debris monitor 56 communicates wear debris information through an analog and/or digital communication interface to a communication controller 76 for signal processing and communications.

Referring to FIGS. 2A-2B, lubricant condition monitor 58 performs oil condition assessment of lubricant in main gearbox 16 through a transducer 72 in order to detect and classify lubricant quality factors such as water content, incorrect lubricant addition, lubricant oxidation degradation, additive depletion, or the like. Lubricant condition monitor 58 may include one or more of a sensing element (e.g., transducer 72), analog circuitry, analog-to-digital converter(s), and/or digital processing circuitry.

Lubricant condition monitor 58 performs lubricant condition assessment of the “filtered” lubricant from gearbox filter 24 (shown in phantom) as it exits gearbox filter 24 and traverses back to proximal end 30 to main gear box 16 (FIG. 1) via central bore 64. The lubricant condition assessment system in lubricant condition monitor 58 uses a low-powered Alternating Current (“AC”) electrochemical impedance spectroscopy (“EIS”) to extract features from the lubricant as it flows through bore 64 as it exits filter 24. In an example, lubricant condition monitor 58 uses a transducer 72 to measure changes in the electrochemical response of the lubricant and estimates the change in specific aspects of lubricant health through a lubricity impedance model. The system electrochemically models the lubricant as a Randles circuit to assess changes in the dielectric properties and conductivity and fluid impedance of the lubricant as it degrades by aging (due to additive depletion, varnish accumulation, oxidation, or the like) or the presence of contaminants such as water or an incorrect lubricant. The lubricant condition monitor 58 injects a multi-frequency AC voltage signal into the lubricant and measures the response at the frequency of the injected signal. The impedance of the lubricant can then be determined by comparing the differences between the injected signal and the response signal. In order to generate injection signals and process the received signals, the lubricant condition monitor 58 includes a condition controller 74 that is in communication with transducer 72. Condition controller 74 may be implemented as a microcontroller, DSP, microprocessor or similar device and includes a memory. The memory may store a lubricant quality algorithm as executable instructions and models for interrogation and analysis of the received signal in order to detect and classify lubricant quality factors in the lubricant. Also, condition controller 74 may communicate information through an analog and/or digital communication interface to communication controller 76 for signal processing and communications.

Communication controller 76 may be implemented as a microcontroller, DSP, microprocessor or similar device and includes a memory. Communication controller 76 includes analog and/or digital communications for high-level digital communication with debris monitor 56 and lubricant condition monitor 58 as well as diagnostic and prognostic algorithms for processing and analyzing information that is received from debris controller 70 and condition controller 74 and providing on-line communications for prognostics and health monitoring (“PHM”). Data communication includes receiving data signals related to wear debris detection and lubricant condition assessment from debris monitor 56 and lubricant condition monitor 58, respectively. Communication controller 76 includes signal processing and analysis of received data signals from debris monitor 56 and lubricant condition monitor 58 and includes one or more algorithms for PHM as well as communicating the processed information on-line to external interfaces. In an embodiment, communications controller 76 can process digital data received from controllers 70, 74 and provide this information to an external interface upon interrogation of the communication controller 76.

As shown in FIG. 2C, housing 50 includes a debris capture port 66 for capturing wear debris particles in lubricant as it flows from gearbox 16 (FIG. 2A) from proximal end 30 into a second pathway 68. Pathway 68 provides a separate and parallel “dirty” lubricant path from gearbox 16 (FIG. 1) into LUCAS apparatus 12 in order to prevent any blockage in the debris capture mechanism from starving the gearbox 16 (FIG. 2A) of lubricant. Debris capture port 66 includes a particle capture element. An exemplary particle capture element comprises a strainer mesh 69 that resides within debris capture port 66 and also within pathway 68. Strainer mesh 69, having a grid like structure, provides an uninterrupted flow of lubricant through pathway 68 but collects suspended wear debris within the lubricant flow as it flows from proximal end 30 to distal end 40 through pathway 68 and into a connected filter 24 (shown in phantom). It is to be appreciated that wear debris monitor 56 performs wear debris detection on the initial “dirty” lubricant as it exits main gearbox 16. As wear debris particles travel to debris capture port 66, strainer mesh 69 provides a coarse debris capture straining element. An additional benefit includes minimizing noise in the acquired data from the transducers/sensors in order to provide a more reliable assessment of lubricant condition.

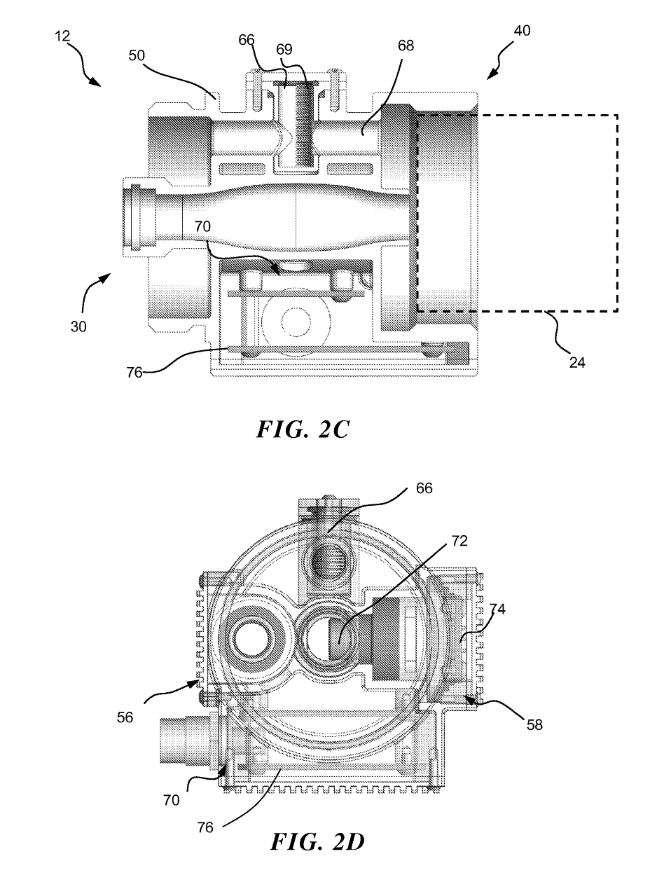

FIG. 3 depicts an exemplary impedance signal of a 100 micrometer iron particle as detected using debris monitor 56. The electromagnetic inductance is represented in the real component 302 of the impedance signal; the magnetic flux reluctance is represented in the imaginary component 304 of the impedance signal. As shown, the phase relationship of the two parts of the impedance signal varies with the material type, which enables the type determination capability of the debris monitor 56.

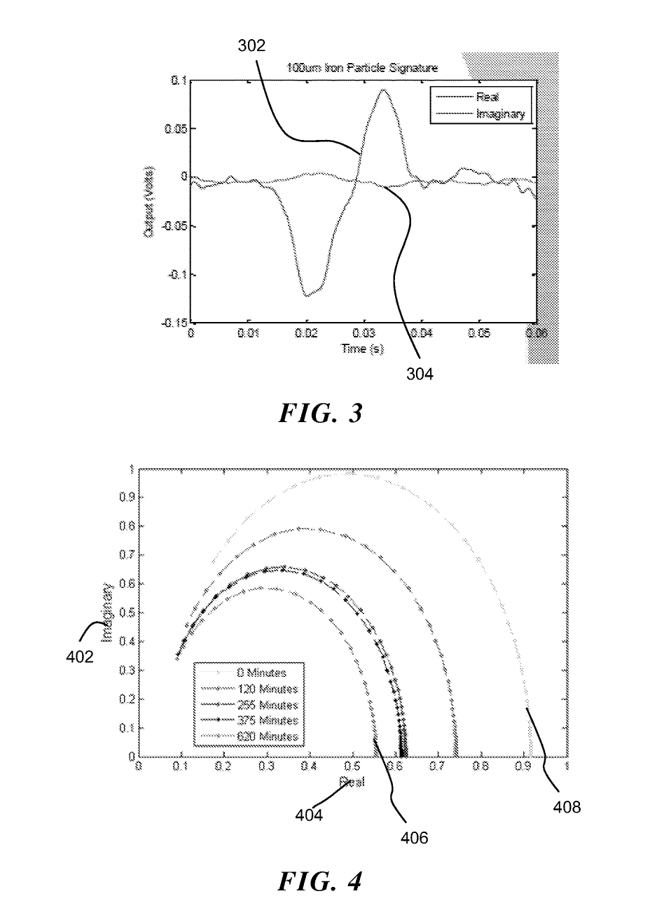

FIG. 4 depicts an exemplary Nyquist plot of oil degradation as measured using EIS through LUCAS apparatus 12. The plot depicts complex impedance of oil as it is oxidizes over controlled conditions with a copper catalyst over time until the sample oil has no further capacity to react (i.e., fully oxidized) using the method described with respect to the lubricant condition monitor 58. The vertical scale 402 is the imaginary impedance value while the horizontal scale 404 is the real impedance value. The smallest curve 406 shows an impedance of fresh oil and the largest curve 408 shows fully oxidized oil. The EIS measures a change in the impedance of the oil as it is fully oxidized and is measurable as a trend from fresh to fully degraded. Depending on how the real and imaginary impedance change over time, the LUCAS apparatus 12 can determine whether a certain lubricant quality factor such as, for example, water contamination, fuel contamination, or degradation of an additive package, is a cause.

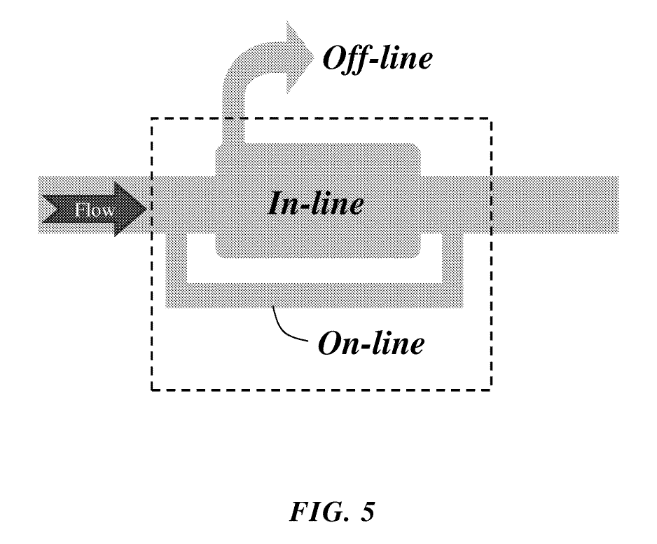

FIG. 5 depicts exemplary lubricant condition assessment topologies for use with the lubricant condition assessment apparatus. In one topology, referred to as an in-line flow path, all lubricant from the gearbox passes through the LUCAS apparatus 12 for inspection by the debris monitor 56 and the lubricant condition monitor 58. In an alternate topology, referred to as an on-line flow path, a portion of the lubricant is diverted from the full flow path, passes through the LUCAS apparatus 12, and then returns to the full flow path. This on-line flow path topology may be implemented as part of a “kidney loop” which includes additional filtering of the lubricant. In an alternate topology, referred to as an off-line flow path, lubricant is removed from the system and passed through the LUCAS apparatus 12 for analysis, for example at a test station. The off-line flow path may also be part of a “kidney loop” which includes additional filtering of the lubricant.

The terminology used herein is for the purpose of describing particular embodiments only and is not intended to be limiting of the invention. While the description of the present invention has been presented for purposes of illustration and description, it is not intended to be exhaustive or limited to the invention in the form disclosed. Many modifications, variations, alterations, substitutions or equivalent arrangements not hereto described will be apparent to those of ordinary skill in the art without departing from the scope and spirit of the invention. Additionally, while the various embodiments of the invention have been described, it is to be understood that aspects of the invention may include only some of the described embodiments. Accordingly, the invention is not to be seen as limited by the foregoing description, but is only limited by the scope of the appended claims.

1. An apparatus for assessment of a fluid system, apparatus comprising:
a debris monitor to receive a first flow of a fluid, the debris monitor to determine wear debris information in the first flow of the fluid; and
a fluid condition monitor to receive a second flow of the fluid, the fluid condition monitor to determine fluid condition information in the second flow of the fluid.

2. The apparatus of claim 1, wherein the debris monitor comprises a sensing element that obtains the wear debris information, the sensing element comprising one or more of an inductive coil, an optical sensing element, a magnetic sensing element and an acoustical sensing element.

3. The apparatus of claim 2, wherein the sensing element comprises the inductive coil, the debris monitor to identify wear debris particles in the fluid by analyzing real and imaginary impedance shifts in magnetic and electric field lines.

4. The apparatus of claim 1, further comprising a communication controller to provide communication of at least one of the wear debris information and the fluid condition information to an external interface.

5. The apparatus of claim 1, wherein the first flow and the second flow are a same flow.

6. The apparatus of claim 1, wherein the debris monitor and the fluid condition monitor are positioned in at least one of an in-line flow path, an on-line flow path and an off-line flow path.

7. The apparatus of claim 1, further comprising a housing, the housing comprising a first flange at a first end and a second flange at a second end, the first flange to couple the housing to the fluid system, the second flange to couple a filter to the housing.

8. The apparatus of claim 1, further comprising a pathway to receive a third flow of the fluid, the pathway comprising a particle capture element in fluidic communication with the third flow of the fluid, the particle capture element to provide a sample of wear debris particles in the third flow of the fluid.

9. The apparatus of claim 8, wherein the third flow of the fluid is parallel to at least one of the first flow of the fluid and the second flow of the fluid.

10. The apparatus of claim 1, wherein the fluid condition monitor is configured to determine at least one of water content, incorrect lubricant addition, lubricant oxidation degradation, additive depletion, or viscosity.

11. The apparatus of claim 1, wherein the fluid is a lubricant from a gearbox of a vehicle.

12. The apparatus of claim 11, wherein the fluid condition information comprises at least one of dielectric properties, conductivity, and fluid impedance.

13. The apparatus of apparatus of claim 1, wherein the debris monitor comprises at least one of analog circuitry, an analog-to-digital converter, and digital processing circuitry.

14. The apparatus of apparatus of claim 1, wherein the fluid condition monitor comprises at least one of analog circuitry, an analog-to-digital converter, and digital processing circuitry.
